(19) United States
(12) Patent Application Publication
(10) Pub. No.: US 2003/0190365 A1
(43) Pub. Date: Oct. 9, 2003

(54) Methods for wet granulating azithromycin

(75) Inventors: Michael Fergione, Stonington, CT (US); Barbara A. Johnson, Niantic, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/327,383

(22) Filed: Dec. 20, 2002

Related U.S. Application Data
(60) Provisional application No. 60/343,469, filed on Dec. 21, 2001.

(51) Int. Cl. A61K 31/7052; B29B 9/00; A61K 9/48; A61K 9/26; A61K 9/14

(52) U.S. Cl. 424/489; 424/452; 424/470; 514/28; 264/5

(57) ABSTRACT

The present invention relates to a method of forming non-dihydrate azithromycin granules, comprising mixing non-dihydrate azithromycin particles, with a granulating amount of a granulating liquid, and, optionally, with one or more excipients, to form wet granules which comprise non-dihydrate azithromycin and the granulating liquid. The granules are then dried to remove the granulating liquid.

The invention further relates to a pharmaceutical composition comprising granules of a non-dihydrate azithromycin and at least one pharmaceutically acceptable excipient.

The invention also relates to pharmaceutical formulations comprising granules of non-dihydrate azithromycin.

The invention further relates to granules of dihydrate azithromycin wherein the granules comprises 98-100% dihydrate azithromycin and from about 0-2%, total weight, of one or more pharmaceutically acceptable excipients.

### BACKGROUND OF THE INVENTION

Granulation is a process whereby granules are formed from a bulk drug substance with or without excipients to improve the properties of the bulk drug or formulation. Granules are preparations consisting of solid, dry agglomerates of powder particles sufficiently robust to withstand handling. Granules usually contain one or more active ingredients with or without auxiliary substances. Granules can either be used as a medicinal form or in the manufacturing process of tablets and capsules, taking advantage of their better compactability, flowability, and limited dust formation. Granules can be enlarged through moist granulation processes such as wet granulation.

Wet granulation is distinguished from dry granulation in that a granulating liquid, such as water, organic liquids or mixtures thereof, are used in wet granulation to produce granules. The advantages of wet granulation include improvement of the cohesiveness and compactability of powders, increase in density, good distribution providing uniform content of micronized or finely milled low-dosage drugs, reduction of dust and airborne contamination, and prevention of segregation of components.

Azithromycin, or 9-deoxo-9a-aza-9a-methyl-9a-homoerythromycin A, is a broad spectrum antibacterial compound derived from erythromycin A.

Azithromycin can be produced in many different forms. For example, the current commercial form of azithromycin is a stable crystalline, non-hygroscopic dihydrate, also referred to herein as form A, which is made according to the method described in U.S. Pat. No. 6,268,489. The commercial tablet is then formulated by wet granulating the dihydrate using water as the granulating liquid.

Several crystalline, non-dihydrate forms of azithromycin are also known. For example, U.S. Pat. No. 4,474,768 discloses a hygroscopic crystalline hydrate of azithromycin which is also referred to herein as form B. This form of azithromycin is difficult to handle during formulation due to its propensity for readily adsorbing varying amounts of water.

It would be desirable to form granules of non-dihydrate forms of azithromycin by wet granulation.

### SUMMARY OF THE INVENTION

The present invention relates to a method of forming non-dihydrate azithromycin granules, comprising mixing non-dihydrate azithromycin particles, with a granulating amount of a granulating liquid, and, optionally, with one or more excipients, to form wet granules which comprise non-dihydrate azithromycin and the granulating liquid. The granules are then dried to remove the granulating liquid.

In the method of the invention, the non-dihydrate azithromycin is selected from azithromycin forms B, D, E, G, H, J, M, N, O, P, Q, R and mixtures thereof. Alternately, the azithromycin is form F.

The invention further relates to a pharmaceutical composition comprising granules of a non-dihydrate azithromycin and optionally at least one pharmaceutically acceptable excipient.

The invention additionally relates to pharmaceutical formulations comprising granules of non-dihydrate azithromycin.

The invention further relates to granules of dihydrate azithromycin wherein the granules comprise 98-100% dihydrate azithromycin and 0-2% , total weight, of one or more pharmaceutically acceptable excipients. DETAILED DESCRIPTION

All percentages discussed herein, unless otherwise noted, are to be considered percentages by weight.

The present invention relates to granules of azithromycin formed by aqueous and nonaqueous-based wet granulation. Preferably, the azithromycin is crystalline. Alternatively, the azithromycin may be non-crystalline or amorphous.

It is also preferable that the azithromycin is non-dihydrate azithromycin. More preferably, the azithromycin is crystalline, non-dihydrate azithromycin.

In the present invention, “granules” are defined as particles of azithromycin and, optionally, particles of at least one excipient, which are adhered together or agglomerated.

Particles, as defined herein, include non-dihydrate azithromycin powder, pharmaceutically acceptable excipient powder, or granules which were previously formed from a non-dihydrate azithromycin powder and, optionally, at least one pharmaceutically acceptable excipient.

Non-dihydrate azithromycin means all amorphous and crystalline forms of azithromycin, including all polymorphs, isomorphs, clathrates, salts, solvates and hydrates thereof, other than the dihydrate form of azithromycin (form A).

Several crystalline, non-dihydrate forms of azithromycin, including forms D, E, F, G, H, J, M, N, O, P, Q and R, are disclosed in U.S. patent application Ser. No. 10/152,106, filed May 21, 2002, the teachings of which are incorporated herein, by reference, in their entirety.

In one embodiment of the present invention, granules are prepared from (1) a non-dihydrate form of azithromycin, selected from forms B, D, E, G, H, J, M, N, O, P, Q and R, or mixtures thereof, and (2) optionally, one or more pharmaceutically acceptable excipients. These forms of non-dihydrate azithromycin are defined as follows.

Both Family I and Family II isomorphs are hydrates and/or solvates of azithromycin. The solvent molecules in the cavities have a tendency to exchange between solvent and water under specific conditions. Therefore, the solvent/water content of the isomorphs may vary to a certain extent. Forms B, F, G, H, J, M, N, O, and P belong to Family I azithromycin and belong to a monoclinic P2₁ space group with cell dimensions of a=16.3±0.3 Å, b=16.2±0.3 Å, c=18.4±0.3 Å and beta=109±2°. Forms D, E and R belong to Family II azithromycin and belong to an orthorhombic P2₁ 2₁2₁ space group with cell dimensions of a=8.9±0.4 Å, b=12.3±0.5 Å and c=45.8±0.5 Å. Form Q is distinct from Families I and II.

Form D azithromycin is of the formula C₃₈H₇₂N₂O₁₂·H₂O·C₆H₁₂ in its single crystal structure, being azithromycin monohydrate monocyclohexane solvate. Form D is further characterized as containing 2-6% water and 3-12% cyclohexane by weight in powder samples. From single crystal data, the calculated water and cyclohexane content of form D is 2.1 and 9.9%, respectively.

Form E azithromycin is of the formula C₃₈H₇₂N₂O₁₂·H₂O·C₄H₈O being azithromycin monohydrate mono-tetrahydrofuran solvate. Form E is a monohydrate and mono-THF solvate by single crystal analysis.

Form G azithromycin is of the formula C₃₈H₇₂N₂O₁₂·1.5H₂O in the single crystal structure, being azithromycin sesquihydrate. Form G is further characterized as containing 2.5-6% water and <1 % organic solvent(s) by weight in powder samples. The single crystal structure of form G consists of two azithromycin molecules and three water molecules per asymmetric unit. This corresponds to a sesquihydrate with a theoretical water content of 3.5%. The water content of powder samples of form G ranges from about 2.5 to about 6%. The total residual organic solvent is less than 1% of the corresponding solvent used for crystallization.

Form H azithromycin is of the formula C₃₈H₇₂N₂O₁₂·H₂O·0.5C₃H₈O₂ being azithromycin monohydrate hemi-1,2 propanediol solvate. Form H is a monohydrate/hemi-propylene glycol solvate of azithromycin free base.

Form J azithromycin is of the formula C₃₈H₇₂N₂O₁₂·H₂O·0.5C₃H₇OH in the single crystal structure, being azithromycin monohydrate hemi-n-propanol solvate. Form J is further characterized as containing 2-5% water and 1-5% 1-propanol by weight in powder samples. The calculated solvent content is about 3.8% n-propanol and about 2.3% water.

Form M azithromycin is of the formula C₃₈H₇₂N₂O₁₂·H₂O·0.5C₃H₇OH, being azithromycin monohydrate hemi-isopropanol solvate. Form M is further characterized as containing 2-5% water and 1-4% 2-propanol by weight in powder samples. The single crystal structure of form M would be a monohydrate/hemi-isopropranolate.

Form N azithromycin is a mixture of isomorphs of Family I. The mixture may contain variable percentages of isomorphs, F, G, H, J, M and others, and variable amounts of water and organic solvents, such as ethanol, isopropanol, n-propanol, propylene glycol, acetone, acetonitrile, butanol, pentanol, etc. The weight percent of water can range from 1-5.3% and the total weight percent of organic solvents can be 2-5% with each solvent content of 0.5 to 4%.

Form O azithromycin is of the formula C₃₈H₇₂N₂O₁₂·0.5H₂O·0.5C₄H₉OH, being a hemihydrate hemi-n-butanol solvate of azithromycin free base by single crystal structural data.

Form P azithromycin is of the formula C₃₈H₇₂N₂O₁₂·H₂O·0.5C₅H12O being azithromycin monohydrate hemi-n-pentanol solvate.

Form Q azithromycin is of the formula C₃₈H₇₂N₂O₁₂·H₂O·0.5C₄H₈O being azithromycin monohydrate hemi-tetrahydrofuran solvate. It contains about 4% water and about 4.5% THF.

Form R azithromycin is of the formula C₃₈H₇₂N₂O₁₂·H₂O·C₅H12O being azithromycin monohydrate mono-methyl tert-butyl ether solvate. Form R has a theoretical water content of 2.1 weight % and a theoretical methyl tert-butyl ether content of 10.3 weight %.

In an alternate embodiment of the present invention, granules are prepared from (1) azithromycin form F, and (2) optionally, one or more pharmaceutically acceptable excipients. Form F azithromycin is of the formula C₃₈H₇₂N₂O₁₂·H₂O·0.5C₂H₅OH in the single crystal structure, being azithromycin monohydrate hemi-ethanol solvate. Form F is further characterized as containing 2-5% water and 1-4% ethanol by weight in powder samples. The single crystal of form F is crystallized in a monoclinic space group, P2₁ with the asymmetric unit containing two azithromycins, two waters, and one ethanol, as a monohydrate/hemi-ethanolate. It is isomorphic to all Family I azithromycin crystalline forms. The theoretical water and ethanol contents are 2.3 and 2.9%, respectively.

In yet another embodiment of the present invention, granules comprise at least about 98% azithromycin form A, and about 2% to 0% of one or more pharmaceutically acceptable excipients. This embodiment is further exemplified by Example 1.

In the method of the present invention, a granulating liquid is defined as a liquid which, when mixed with the azithromycin, and optional excipient particles, promotes adherence, or agglomeration, of the particles to form granules.

A granulating amount of a granulating liquid is an amount of liquid sufficient to permit particle adherence, or agglomeration, without significant dissolution of the azithromycin.

Granulating liquids of the present invention may be nonaqueous or aqueous.

A nonaqueous granulating liquid is defined herein as an organic solvent which contains 25% or less, by volume, water. Suitable organic solvents include, but are not limited to, acetonitrile, chlorobenzene, chloroform, cyclohexane, 1,2-dichlorethane, dichloromethane, 1,2-dimethoxyethane, N,N,-dimethylacetamide, N,N-dimethylformamide, 1,4 dioxane, 2-ethoxyethanol, ethylene glycol, formamide, hexane, 2-methoxyethanol, methylbutyl ketone, methylcyclohexane, N-methyl-pyrrolidone, nitromethane, pyridine, sulfolane, tetralin, toluene, 1,2-trichlorethane, xylene, acetic acid, acetone, anisole, butyl acetate, tert-butylethylether, cumene, dimethyl sulfoxide, ethyl acetate, ethyl ether, ethyl formate, formic acid, heptane, isobutyl acetate, isopropyl acetate, methyl acetate, methylethyl ketone, methylisobutyl ketone, pentanel, propyl acetate, tetrahydrofuran, C₁-C₆ alcohols, and mixtures thereof.

The nonaqueous granulating liquid may also be a miscible mixture of one or more organic solvents and/or water.

Preferred nonaqueous granulating liquids of the present invention include ethanol, isopropanol, and miscible mixtures thereof with water, which are further described in Examples 1-8 herein. For nonaqueous granulating liquids, ethanol is preferred for granulating form F. Isopropanol is preferred for granulating form M.

An aqueous granulating liquid, as defined herein, is a granulating liquid comprising more than 25% water and less than 75% of one or more suitable organic solvents as specified above. A preferred aqueous granulating liquid, of the present invention, is a miscible mixture of water and ethanol, which is further described in Examples 1-8 herein.

As defined herein, the term “pharmaceutically acceptable” means that the excipient must be compatible with other ingredients of the composition, and not deleterious to the recipient thereof.

Pharmaceutically acceptable excipients, of the present invention, include binders, diluents, disintegrants, lubricants, fillers, carriers, and the like. Further, the excipients may be hygroscopic or non-hygroscopic.

Binders are used to impart cohesive qualities to a granulation, and thus ensure that a granulation remains intact after drying and milling. They are also important for providing granule particle size uniformity and compaction properties of the granulation. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia, sodium alginate, polyvinylpyrrolidone, cellulosic polymers (including hydroxypropyl cellulose, hydroxypropyl methylcellulose, methyl cellulose, hydroxyethyl cellulose, and the like). For aqueous granulating solutions, preferred binders include hydroxypropyl cellulose, polyvinylpyrrolidone, pregelatinized starch, and sugar, for example sucrose.

Lubricants can be employed herein in the manufacture of certain dosage forms, and will usually be employed when producing tablets. Typically, the lubricant is added just before the tableting step, and is mixed with the granulate for a short period of time to obtain good dispersal. Typical mixing times are in the range of about five minutes. The lubricant employed in a composition of the present invention may be one or more compounds. Examples of suitable lubricants include, but are not limited to, magnesium stearate, calcium stearate, zinc stearate, stearic acid, talc, glyceryl behenate, polyethylene glycol, polyethylene oxide polymers (for example, available under the registered trademarks of Carbowax™ for polyethylene glycol and Polyoxm for polyethylene oxide from Dow Chemical Company, Midland, Mich.), sodium lauryl sulfate, magnesium lauryl sulfate, sodium oleate, sodium stearyl fumarate, DL-leucine, colloidal silica, and others as known in the art. Preferred lubricants are magnesium stearate, calcium stearate, zinc stearate and mixtures of magnesium stearate with sodium lauryl sulfate. Lubricants may comprise from about 0.25 to about 10% of the tablet weight, preferably from about 0.25 to about 3% for the preferred lubricants.

Suitable diluents may be one or more compounds which are capable of providing compactability and good flow. A variety of materials may be used as fillers or diluents. Suitable diluents or fillers include, but are not limited to, spray-dried monohydrate or anhydrous lactose, sucrose, dextrose, mannitol, sorbitol, starch, cellulose (e.g. microcrystalline cellulose; Avicel®, FMC Biopolymer, Philadelphia, Pa.), dihydrated or anhydrous dibasic calcium phosphate (available commercially under the registered trademark Emcompress® from Penwest Pharmaceuticals Co., Cedar Rapids, Iowa or A-Tab® and Di-Tab® from Rhodia Inc, Cranbury, N.J.), calcium carbonate, calcium sulfate, and others as known in the art.

In the present invention, disintegrants may be added intragranularly and/or extragranularly. Disintegrants are used to facilitate tablet disintegration or “breakup” after administration, and are generally starches, clays, celluloses, algins, gums or crosslinked polymers. Suitable disintegrants include, but are not limited to, crosslinked polyvinylpyrrolidone (PVP-XL), sodium starch glycolate, and croscarmellose sodium.

If desired, the granule or pharmaceutical composition may also contain minor amounts of nontoxic auxiliary substances such as wetting or emulsifying agents, pH buffering agents and the like, for example, sodium acetate, sorbitan monolaurate, triethanolamine, sodium acetate, triethanolamine oleate, sodium lauryl sulfate, dioctyl sodium sulfosuccinate, polyoxyethylene sorbitan fatty acid esters, etc.

In the method for forming granules containing non-dihydrate azithromycin, non-dihydrate azithromycin powder is mixed with a granulating amount of a suitable granulating liquid to form good granules within the granule/granulating liquid mixture, which is hereinafter referred to as the “wet granulate”.

Good granules typically have few fines, uniform size and stay intact after drying and sizing. Sizing may be accomplished by a sieve or mill, for instance. The skilled worker often makes a subjective determination by observing the consistency of the granules.

In an alternate embodiment, the granulating liquid is mixed with the non-dihydrate azithromycin particles and with particles of at least one excipient, to form granules. These granules are then dried, by suitable means, to form a pharmaceutical composition which comprises granules containing non-dihydrate azithromycin and the pharmaceutically acceptable excipients.

Optionally, the azithromycin and excipients may be preblended prior to mixing with the granulating liquid. Preblending can be accomplished by blending, mixing, stirring, shaking, tumbling, rolling or by any other method to achieve a homogeneous blend. It is preferred that the azithromycin and excipients be combined under low shear conditions in a suitable apparatus, such as a V-blender, tote blender, double cone blender or any other apparatus capable of functioning under preferred low shear conditions.

In yet another embodiment, the non-dihydrate azithromycin particles that are to be mixed with the granulating liquid, and optional excipients, is in the form of previously granulated azithromycin particles. Further, these previously granulated azithromycin particles may further include, intragranularly, one or more pharmaceutically acceptable excipients.

In the method of the present invention, the particles are mixed with the granulating liquid for a period from about 5 to about 45 minutes. Preferably, for production scale, the mixing time is about 20 to about 35 minutes. For small scale, the mixing time is preferably about 3 minutes to about 10 minutes. Also, wet granulation is generally performed at temperatures between about 20° C. to about 30° C., and preferably about room temperature.

Any equipment may be used to contact the granulating liquid with the particles as long as uniform distribution of the granulating liquid and good contact of the particles are achieved. For example, small-scale production can be achieved by mixing and wetting the mass in mortars or stainless steel bowls, while for larger quantities V-blenders with intensifier bars, planetary mixers, rotary granulators, high shear mixers and fluid-bed granulation equipment may be used.

The extent of granule formation may be determined by visual observation and manual manipulation, as is common in the art. The extent of granule formation may also be determined by sieve analysis, moisture measurements, such as loss on drying (LOD) or other suitable methods, such as instrumented endpoint analysis using measurements of torque and power consumption.

The choice of a particular granulating liquid system depends on a number of factors, such as the form of azithromycin being used, and may be based on desired processing characteristics. For example, it was found that the different crystalline forms of azithromycin had differences in solubility profiles in different solvents. For example, form A exhibited much lower solubility in water and isopropanol solutions as compared to the other forms. However, in ethanol, all the crystalline forms examined appeared to have similar solubilities.

Additionally, in many organic-based granulating liquids, for example ethanol/water, isopropanol/water, isopropanol, and ethanol, azithromycin granules can be formed without the inclusion of an excipient, in particular a binder. Thus, a higher drug loading may be obtained when a binder is not used.

Conversely, a binder is preferred for azithromycin granulation with water.

However, for water, residual water can be removed from the granules by drying using readily available equipment, while processes involving the use of organic solvents may require additional solvent removal steps.

In the present method, it is preferred that azithromycin be wet granulated in a manner according to the guidelines set by the International Conference on Harmonization of Technical Requirements for Registration of Pharmaceuticals for Human Use in the publication Harmonized Tripartite Guideline: Impurities: Guideline for Residual Solvents, recommended for adoption on Jul. 17, 1997.

Following granule formation, the granules in the wet granulate are then dried by suitable means to remove the granulating liquid. The conditions and duration of drying depend on the liquid used and the mass of material. Examples of suitable drying methods include, but are not limited to, tray drying, forced air drying, microwave drying, vacuum drying and fluid bed drying. Optionally, the wet granulate may be sized before drying. Suitable sizing operations for the wet granulate include wet milling or sieving.

Typically, the pharmaceutical formulation that is contacted with the granulating liquid comprises from about 30 to about 98%, more preferably from about 50 to about 60% of azithromycin, by weight, and at least one excipient.

Suitable pharmaceutical formulations for wet granulation may comprise from about 15% to about 98% azithromycin, from about 0.25% to about 84.5% binder, preferably from about 0.5% to about 30% binder, more preferably from about 0.5% to about 6% binder and from about 0% to about 80% filler and from about 0.5% to about 25% disintegrant, more preferably from about 0.5% to about 15% disintegrant, most preferably from about 1% to about 6% disintegrant.

If a binder is used, it may be dissolved in the aqueous or nonaqueous granulating liquid. If dissolved in the granulating liquid, the binder may be used in amounts of from about 0.45% to about 25% (weight/volume of liquid), more preferably in amounts of from about 5% to about 10% (weight/volume). Alternatively, the binder in its dry form may be incorporated into the powder prior to granulation. If incorporated into the powder prior to granulation, the binder may be used in amounts of from about 0.25% to about 85% by weight, based on the weight of powder, preferably in an amount of from about 0.5% to about 30% by weight, based on the weight of the powder, more preferably in an amount of from about 0.5% to about 6% by weight, based on the weight of the powder. The particular weight percentage of the binder will depend on the particular binder chosen, as will be recognized by the skilled formulator. Alternatively, the binder may be included in both the granulating liquid and the powder.

The amount of granulating liquid used in preparing granulations will vary depending on the granulating liquid and drug form.

In the method of the present invention, for forming granules of non-dihydrate azithromycin, the amount of granulating liquid used (expressed as a percentage of dry weight of the powder) to form good granules will vary with the drug loading, whether or not the azithromycin is form F, whether a hygroscopic excipient is included, and whether the liquid is aqueous or nonaqueous.

For example, the incorporation of hygroscopic excipients, such as croscarmellose sodium, require larger amounts of aqueous granulating liquid. Hygroscopic excipients are defined as those excipients which are significantly hygroscopic, absorbing more than about 20% moisture at moderate relative humidities of 35-50%, such as croscarmellose sodium, A. H. Kibbe, ed. Handbook of Pharmaceutical Excipients third edition, American Pharmaceutical Association, 2000. The type of equipment used in processing will also have an influence on the amount of granulating liquid used. For example, high shear equipment and larger scale equipment typically require less liquid for granulation.

Examples of non-hygroscopic excipients are: sodium starch glycolate, polyvinylpyrrolidone, crosslinked PVP (PVP-XL), and hydroxypropylcellulose.

Further, granulation of form F azithromycin requires more granulating liquid than do other forms of non-dihydrate azithromycin.

Based upon these variables, suitable embodiments for granulating different non-dihydrate forms of azithromycin are provided, as follows.

In wet granulations, with an aqueous granulating liquid, wherein non-dihydrate forms of azithromycin, excluding form F, at drug loadings of from about 30% to about 98%, and wherein no hygroscopic excipients are included intragranularly, the amount of aqueous granulating liquid is typically in the range of about 10% to about 30% and is preferably between about 10% to about 20%.

In wet granulations, with an aqueous granulating liquid, wherein non-dihydrate forms of azithromycin, excluding form F, at drug loadings of from about 30% to about 98%, and hygroscopic excipients are included intragranularly, the amount of aqueous granulating liquid is typically in the range of about 18% to about 45% and is preferably between about 30% to about 40%.

In wet granulations, with a nonaqueous granulating liquid, wherein non-dihydrate forms of azithromycin, excluding form F, at drug loadings of from about 30% to about 98%, the amount of nonaqueous granulating liquid is typically in the range of about 7.5% to about 50% and is preferably between about 10% to about 20%.

In wet granulations, with an aqueous granulating liquid, using azithromycin form F, at drug loadings of from about 30% to about 98%, and wherein no hygroscopic excipients are included intragranularly, the amount of aqueous granulating liquid is typically in the range of about 20% to about 40% and is preferably between about 25% to about 35%.

In wet granulations, with an aqueous granulating liquid, using azithromycin form F, at drug loadings of from about 30% to about 98%, and hygroscopic excipients are included intragranularly, the amount of aqueous granulating liquid is typically in the range of about 30% to about 55% and is preferably between about 40% to about 50%.

In wet granulations, with a nonaqueous granulating liquid, using azithromycin form F, at drug loadings of from about 30% to about 98%, the amount of nonaqueous granulating liquid is typically in the range of about 10% to about 55% and is preferably between about 20% to about 30%.

For embodiments wherein greater than about 50% drug loadings are used, for all azithromycin forms, using granulating liquids comprising less than 50% water generally produce better granules. In embodiments wherein 100% azithromycin loading is used, granulating liquids comprising less than 50% water are preferred and nonaqueous granulating liquids containing 5% or less water are more preferred.

More specifically, the amount of nonaqueous granulating liquid for high azithromycin loadings (specifically greater than 98%) of azithromycin forms, excluding form F, is typically from about 10% to about 25%, and preferably from about 15% to about 20%.

For form F, the amount of nonaqueous granulating liquid for high azithromycin loadings (specifically greater than 98%) of azithromycin form F is typically between about 20% to about 40%, and more preferably between about 25% to about 35%.

Also, the amount of aqueous granulating liquid for high azithromycin loadings (specifically greater than 98%) of azithromycin forms, excluding form F, is typically from about 15% to about 30%, and preferably from about 17% to about 25%.

For form F, the amount of aqueous granulating liquid for high azithromycin loadings (specifically greater than 98%) of azithromycin form F is typically between about 40% to about 60%, and more preferably between about 45% to about 55%.

The pharmaceutical compositions, and granules, of the present invention, optionally, include, intragranularly or extragranularly, additional components such as antioxidants, suspending agents, thickening agents, and the like. The term “extragranular” or “extragranularly” as used herein means that the referenced material is added or has been added as a dry component after granulation. The term “intragranular” or “intragranularly” as used herein means that the referenced material is added or has been added as a component of the granulation.

Flavors may also be included in the pharmaceutical composition. These flavors may be chosen from synthetic flavor oils and flavoring aromatics and/or natural oils, extracts from plants leaves, flowers, fruits, and so forth and combinations thereof. These flavors may include cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil. Also useful as flavors are vanilla, citrus oil, including lemon, orange, grape, lime and grapefruit, and fruit essences, including apple, banana, pear, peach, strawberry, raspberry, cherry, plum, pineapple, apricot, and so forth. The amount of flavoring may depend on a number of factors including the organoleptic effect desired. In a tablet, the flavoring will typically be present in an amount of from 0.5 wt. % to about 3.0 wt. % based on the total tablet weight.

For sachets and powders for suspension, the preferred flavoring comprises a combination of cherry, banana and vanilla flavors as further described in Table XIII of U.S. Pat. No. 5,605,889. The teachings of U.S. Pat. No. 5,605,889, in their entirety, are incorporated herein by reference.

Other excipients and coloring agents may also be added to azithromycin tablets. Coloring agents include, but are not limited to, titanium dioxide and/or dyes suitable for food such as those known as F. D. & C, dyes, aluminum lakes and natural coloring agents such as grape skin extract, beet red powder, beta carotene, annato, carmine, turmeric, paprika, and so forth. A coloring agent is an optional ingredient in the compositions of this invention, but when used will generally be present in an amount up to about 3.5 percent based on the total tablet weight.

The azithromycin granules, and the pharmaceutical compositions, prepared by the method of this invention may be used to prepare pharmaceutical formulations including, but not limited to, tablets, capsules and sachets used for preparing liquid suspensions of azithromycin.

After drying, the granules may optionally be subjected to additional processing steps including, but not limited to, milling, screening or other sizing steps, addition of lubricants and/or other excipients, tableting or encapsulation.

As mentioned, the granules may optionally be subjected to additional processing steps depending on the desired end-use of the material. Additional processing steps include, but are not limited to milling and compaction to form tablets.

In the pharmaceutical industry, milling is often used to reduce the particle size of solid materials. Many types of mills are available and one of the most commonly used types of mill is the hammer mill. This type of mill uses a high-speed rotor to which a number of hammers are attached. The hammers can be attached such that either the knife face or the hammer face contacts the material. As material is fed into the mill, it impacts on the rotating hammers and breaks up into smaller particle sizes. A screen is located below the hammers, which allows the smaller particles to pass through the openings in the screen. Larger particles are retained in the mill and continue to be broken up by the hammers until the particles are fine enough to flow through the screen. Any suitable equipment for reducing the particle size may be used in the present invention.

If desired, the granules may be processed further to form tablets from milled, sieved, or unmilled material. The term “tablet” as used herein is intended to encompass compressed pharmaceutical dosage forms of all shapes and sizes.

In the present invention, equipment may optionally be used to assist the feeding of the granulation or powder during processing. The granulate or powder may be screw fed by means of an augur or by means of paddles in the feed frame on the tablet press or encapsulation equipment. The means of assisted feeding is not limited to any particular type of equipment, and any equipment known in the art may be used to assist the feed of the powder or granulate.

Tablets may be formed from the granules by compression or by molding. Typical compression techniques utilize a piston like device with three stages in each cycle 1) filling (adding the constituents of the tablet to the compression chamber) 2) compaction (forming the tablet) and 3) ejection (removing the tablet). The cycle is then repeated. In one embodiment, a high-speed rotary tablet press may be used. Examples of suitable high-speed rotary tablet presses include Kilian LX2 (manufactured by IMA-Kilian, Cologne, Germany), Manesty BB4 and Manesty Mark IV (both manufactured by Manesty Machines Ltd., Liverpool, England).

Tablets may contain from about 10% to about 90% by weight of azithromycin, preferably from about 25% to about 80% azithromycin on a percentage basis of the weight of the azithromycin to the total weight of the azithromycin plus excipients. Capsules may contain from about 10% to 100% azithromycin, preferably from about 25% to about 95% azithromycin on a percentage basis of the weight of the azithromycin to the total weight of the azithromycin plus excipients. Sachets and powders for suspension may contain from about 0.5% to about 99% azithromycin, preferably from about 0.75% to about 20% azithromycin, more preferably from about 1% to about 10% azithromycin on a percentage basis of the weight of the azithromycin to the total weight of the azithromycin plus excipients.

Flow of the blend on high-speed tablet presses is very important to good weight control of the tablet. The use of a force feeder often improves tablet weight control for poorer flowing blends. Another common feature of high-speed tablet presses is the ability to use precompression. Precompression gently taps the blend when the die is full with blend and then the main compaction takes place to form the tablet.

In the present invention, the granulation step results in particles that are free flowing and have good characteristics for tableting. The term “free flowing” means ease of handling as in, for example, measuring, introducing into packages, or feeding into tableting or encapsulating equipment. Free flowing materials exhibit low cohesion and have the ability to keep moving consistently under the force of gravity without any applied agitation.

Flow properties of a formulation may be evaluated by a number of methods known in the art. One way of characterizing formulation properties of a powdered material is by bulk density measurements. A simple method to provide a description of flow characteristics by bulk density measurement is Carr's Compressibility Index (Carr's Index). Carr's Compressibility Index is a simple test to evaluate flowability by comparing both the initial and final (tapped) bulk volumes and the rate of packing down. A useful empirical guide to flow is given by Carr's compressibility index:

Compressibility Index (%)=[(tapped density−initial density)/tapped density]×100

It is preferred that the granules, of the present invention, have a Carr's Compressibility Index less than about 34%; more preferably less than about 31%; even more preferably less than about 28%.

The tablets prepared from the granulation of the present invention exhibit acceptable physical characteristics including good friability and hardness. The resistance of a tablet to chipping, abrasion or breakage under conditions of storage and transportation depends on its friability. The desired hardness may vary, depending on factors such as tablet size and shape.

Friability is a standard test known to one skilled in the art. Friability is measured under standardized conditions by weighing out a certain number of tablets (generally 20 or less), placing them in a rotating Plexiglas drum in which they are lifted during replicate revolutions by a radial lever, and then dropped a distance of approximately 8 inches. After replicate revolutions (typically 100 revolutions at 25 rpm), the tablets are reweighed and the percentage of formulation abraded or chipped off is calculated. Friability in the range of about 0% to 3%, more preferably about 0 to 1%, is considered acceptable for most drug and food tablet contexts. Friability, which approaches 0%, is particularly preferred.

In one embodiment, the tablet may be a modified capsule shape containing about 250 mgA, about 450 mg total weight. In one embodiment, the dimensions of the aforementioned tablet are 0.26″×0.53″. The term “mgA” as used herein refers to milligrams of the free base of azithromycin. The tablet hardness may be from about 6 to about 18 kP.

In a further embodiment, the tablet may be a modified oval shape containing about 500 mgA, about 900 mg total weight. In one embodiment, the dimensions of the tablet are 0.33″×0.67″. The tablet hardness may be from about 6 to about 26 kP. In an even further embodiment, the tablet may be a modified oval shape containing about 600 mgA, about 1070 mg total weight. In one embodiment, the dimensions of the aforementioned tablet are 0.41″×0.75″. The tablet hardness may be from about 6 to about 26 kP. A reference to tablet shapes can be found in FIG. 25, page 51 of the Tableting Specification Manual, fourth edition, published by the American Pharmaceutical Association, Washington, D.C., 1995.

The tablet may optionally be coated. The reasons for coating a tablet may include masking the taste of the drug, making tablets easier to swallow, protection against chipping during packaging, a barrier for moisture or light to improve product stability, and enhance product appearance or recognition.

The coating process may include the use of a coating solution or suspension, usually aqueous that has acceptable viscosity for spraying and properties for it to adhere to the surface of the tablet when applied. During the coating process, the coating solution or suspension is atomized into fine droplets that come into contact with the tablet. As the droplets dry, a film is formed on the tablet, which is the coating. There are several types of coating equipment used to coat tablets. One type is the pan coater in which tablets are rotated in a pan and coating solution is applied to the tablets as the tablets tumble in the pan. Another coating process involves suspending the tablets in a column of air while the coating solution is sprayed onto the tablet (fluid bed process). The tablet may be coated by any known process and the manner of application is not limited to any particular equipment.

The tablet coating(s) may be a white or colored Opadry® (Colorcon, West Point Pa.) suspension or a clear Opadry® solution. Alternatively a typical coating formulation would consist of a film forming polymer(s) such as hydroxypropyl methylcellulose (HPMC), hydroxypropyl cellulose (HPC), polyvinylpyrrolidone (PVP) with additional ingredients such as plasticizers, opacifiers, colorants and/or antioxidants.

The pharmaceutical compositions of the present invention may be used for the treatment of bacterial or protozoal infections. The term “treatment”, as used herein, unless otherwise indicated, means the treatment or prevention of a bacterial or protozoal infection, including curing, reducing the symptoms of or slowing the progress of said infection.

As used herein, unless otherwise indicated, the term “bacterial infections” or “protozoal infections” includes bacterial infections and protozoal infections that occur in mammals, fish and birds as well as disorders related to bacterial infections and protozoal infections that may be treated or prevented by administering antibiotics such as the compound of the present invention. Such bacterial infections and protozoal infections and disorders related to such infections include, but are not limited to, the following: pneumonia, otitis media, sinusitis, bronchitis, tonsillitis, and mastoiditis related to infection by Streptococcus pneumoniae, Haemophilus influenzae, Moraxella catarrhalis, Staphylococcus aureus, or Peptostreptococcus spp.; pharynigitis, rheumatic fever, and glomerulonephritis related to infection by Streptococcus pyogenes, Groups C and G streptococci, Clostridium diptheriae, or Actinobacillus haemolyticum; respiratory tract infections related to infection by Mycoplasma pneumoniae, Legionella pneumophila, Streptococcus pneumoniae, Haemophilus influenzae, or Chlamydia pneumoniae; uncomplicated skin and soft tissue infections, abscesses and osteomyelitis, and puerperal fever related to infection by Staphylococcus aureus, coagulase-positive staphylococci (i.e., S. epidermidis, S. hemolyticus, etc.), Streptococcus pyogenes, Streptococcus agalactiae, Streptococcal Groups C-F (minute-colony streptococci), viridans streptococci, Corynebacterium minutissimum, Clostridium spp., or Bartonella henselae; uncomplicated acute urinary tract infections related to infection by Staphylococcus saprophyticus or Enterococcus spp.; urethritis and cervicitis; and sexually transmitted diseases related to infection by Chlamydia trachomatis, Haemophilus ducreyi, Treponema pallidum, Ureaplasma urealyticum, or Neisseria gonorroeae; toxin diseases related to infection by S. aureus (food poisoning and Toxic shock syndrome), or Groups A, B, and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by Chlamydia trachomatis, Neisseria gonorrhoeae, S. aureus, S. pneumoniae, S. pyogenes, H. influenzae, or Listeria spp.; disseminated Mycobacterium avium complex (MAC) disease related to infection by Mycobacterium avium, or Mycobacterium intracellulare; gastroenteritis related to infection by Campylobacter jejuni; intestinal protozoa related to infection by Cryptosporidium spp.; odontogenic infection related to infection by viridans streptococci; persistent cough related to infection by Bordetella pertussis; gas gangrene related to infection by Clostridium perfringens or Bacteroides spp.; and atherosclerosis related to infection by Helicobacter pylori or Chlamydia pneumoniae. Bacterial infections and protozoal infections and disorders related to such infections that may be treated or prevented in animals include, but are not limited to, the following: bovine respiratory disease related to infection by P. haem., P. multocida, Mycoplasma bovis, or Bordetella spp.; cow enteric disease related to infection by E. coli or protozoa (i.e., coccidia, cryptosporidia, etc.); dairy cow mastitis related to infection by Staph. aureus, Strep. uberis, Strep. agalactiae, Strep. dysgalactiae, Klebsiella spp., Corynebacterium, or Enterococcus spp.; swine respiratory disease related to infection by A. pleuro., P. multocida, or Mycoplasma spp.; swine enteric disease related to infection by E. coli, Lawsonia intracellularis, Salmonella, or Serpulina hyodyisinteriae; cow footrot related to infection by Fusobacterium spp.; cow metritis related to infection by E. coli; cow hairy warts related to infection by Fusobacterium necrophorum or Bacteroides nodosus; cow pink-eye related to infection by Moraxella bovis; cow premature abortion related to infection by protozoa (i.e. neosporium); urinary tract infection in dogs and cats related to infection by E. coli; skin and soft tissue infections in dogs and cats related to infection by Staph. epidermidis, Staph. intermedius, coagulase neg. Staph. or P. multocida; and dental or mouth infections in dogs and cats related to infection by Alcaligenes spp., Bacteroides spp., Clostridium spp., Enterobacter spp., Eubacterium, Peptostreptococcus, Porphyromonas, or Prevotella. Other conditions that may be treated by the compounds and preparations of the present invention include malaria and atherosclerosis. Other bacterial infections and protozoal infections and disorders related to such infections that may be treated or prevented in accord with the method and compositions of the present invention are referred to in J. P. Sanford et al., “The Sanford Guide To Antimicrobial Therapy,” 26th Edition, (Antimicrobial Therapy, Inc., 1996).

The term “effective amount” means the amount of azithromycin which, when administered, prevents the onset of, alleviates the symptoms of, stops the progression of, or eliminates a bacterial infection in a mammal.

The term “mammal” is an individual animal that is a member of the taxonomic class Mammalia. The class Mammalia includes, for example, humans, monkeys, chimpanzees, gorillas, cattle, swine, horses, sheep, dogs, cats, mice and rats.

In the present invention, the preferred mammal is a human.

Typically, azithromycin, is administered in dosage amounts ranging from about 0.2 mg per kg body weight per day (mg/kg/day) to about 200 mg/kg/day in single or divided doses (i.e., from 1 to 4 doses per day), although variations will necessarily occur depending upon the species, weight and condition of the subject being treated and the particular route of administration chosen. The preferred dosage amount is from about 2 mg/kg/day to about 50 mg/kg/day.

The azithromycin may be administered orally, or by other known means for administering azithromycin.

### EXEMPLIFICATION

The present invention will be further illustrated by means of the following examples. It is to be understood, however, that the invention is not meant to be limited to the details described therein.

### Example 1

Wet Granulation of Bulk Azithromycin Forms A, G, J, M or N

Wet granulations were prepared from various forms of bulk crystalline azithromycin using both aqueous and non-aqueous granulating liquids. The azithromycin forms used were A, G, J, M and N. The granulating liquids used were ethanol 95% (EtOH), isopropanol (IPA), and water (H₂O).

Samples of 3 to 5 g of each bulk drug were granulated in a 30 cubic centimeter (cc) bottle, using a bent micro-spatula impeller with a ½″ blade on a variable speed mini-drill press (Micro-Drill model 164C-7, Cameron Precision Engineering Co., Sonora, Calif. 95370). Prior to use, the blade was bent to an angle sufficient to sweep the material being granulated, and to allow a portion of this material to flow over the top of the blade. The blade was bent to an angle about 30° from vertical. The blade was bent to model the characteristics of the impeller of a Niro SP-l high shear granulator (Niro Inc., Columbia, Md.). The liquid was pipetted in 0.1 to 0.5 mL increments, wet mixing for 2-3 minutes until a suitable granulation was formed based upon visual observations. All runs formed wet granulations, but those using water as the granulating liquid did not to hold together as well as the non-aqueous runs after drying. All of the samples were dried overnight at 40° C. for 16 hours in a forced hot air dryer. Visual observations of manual granulation manipulation showed that the water granulations tended to be softer and more friable than the non-aqueous granulations. The results of the wet granulation experiment are summarized, below, in Table 1.

TABLE 1

| | Drug | Granulation | Batch | Liquid Amount | Granule |
|---|---|---|---|---|---|
| Run # | Form | Fluid | Size | (%)* | Quality |
| 1 | A | EtOH | 3 g | 0.5 mL (17%) | Good |
| | | | | | Granules |
| 2 | G | EtOH | 3 g | 0.5 mL (17%) | Good |
| | | | | | Granules |
| 3 | M | EtOH | 3 g | 0.5 mL (17%) | Good |
| | | | | | Granules |
| 4 | N | EtOH | 3 g | 0.5 mL (17%) | Good |
| | | | | | Granules |
| 5 | J | EtOH | 5 g | 1.0 mL (20%) | Good |
| | | | | | Granules |
| 6 | A | IPA | 3 g | 0.5 mL (17%) | Good |
| | | | | | Granules |
| 7 | G | IPA | 3 g | 0.5 mL (17%) | Good |
| | | | | | Granules |
| 8 | M | IPA | 3 g | 0.5 mL (17%) | Good |
| | | | | | Granules |
| 9 | N | IPA | 3 g | 0.5 mL (17%) | Good |
| | | | | | Granules |
| 10 | J | IPA | 5 g | 1.0 mL (20%) | Good |
| | | | | | Granules |
| 11 | A | H₂O | 3 g | 0.5 mL (17%) | Poor |
| | | | | | Granules |
| 12 | G | H₂O | 3 g | 0.5 mL (17%) | Poor |
| | | | | | Granules |
| 13 | M | H₂O | 3 g | 0.5 mL (17%) | Poor |
| | | | | | Granules |
| 14 | N | H₂O | 3 g | 0.5 mL (17%) | Poor |
| | | | | | Granules |
| 15 | J | H₂O | 5 g | 1.0 mL (20%) | Fair |
| | | | | | Granules |

*Percent listed is the liquid volume amount compared to the dry component amount

A Scanning Electron Microscope (SEM) examination of azithromycin bulk drug wet granulations (forms A, G, J, M and N) with EtOH, IPA, or H₂O was performed. The magnification range used was 50-1000×. Micrographs were taken on 4″×5″ paper film. Granulation quality was visually accessed by SEM with the results shown below in Table 1A.

TABLE 1A

| | Drug | Solvent | Fines | |
|---|---|---|---|---|
| Run | Form | Used | Reduced | Surface Changes |
| 1 | A | EtOH | Yes | Yes |
| 2 | G | EtOH | Yes | Yes |
| 3 | M | EtOH | Yes | Yes |
| 4 | N | EtOH | Yes | Yes |
| 5 | J | EtOH | Yes | Yes |
| 6 | A | IPA | Yes | Yes |
| 7 | G | IPA | Yes | Yes |
| 8 | M | IPA | Yes | Yes |
| 9 | N | IPA | Yes | Yes |
| 10 | J | IPA | Yes | Yes |
| 11 | A | H₂O | N/S | N/S |
| 12 | G | H₂O | N/S | N/S |
| 13 | M | H₂O | N/S | N/S |
| 14 | N | H₂O | N/S | N/S |
| 15 | J | H₂O | N/S | N/S |

N/S = Not Significant

The SEM's confirmed that good granulations were formed for all five drug forms using both the 95% EtOH and the IPA. The surface of the drug crystals appeared to have dissolved and acted as a binder to help form the granules. The surface appearance was similar to that of a thinly spread coating on the granules. Fines were also reduced from the amount visible in the “as-is” ungranulated bulk drug.

The water also appeared to make a good granulation for all drug forms while still wet, but after drying the granules tended to be very friable, indicating weak binding. Form J made slightly better quality granules than the other forms, but this was probably due to a higher percentage of water added than for the other forms. Indications are that an apparent overwetting with water will make a less friable dried granule, but still of a lower quality than that obtained using non-aqueous granulating liquids. SEM examination indicated that the crystals were similar in appearance to the initial bulks. There was no significant indication of fines being reduced or surface dissolving. It appeared that water alone was a poor choice of granulating liquid for bulk azithromycin without using a binder or additional excipients.

### Example 2

Wet Granulation of Bulk Form F Azithromycin

Wet granulations were prepared from bulk crystalline, non-dihydrate form F azithromycin using both aqueous and nonaqueous granulating liquids. The granulating liquids used were 95% ethanol, isopropanol, and water.

Form F bulk drug contained soft lumps and was sieved through an 18 mesh (1.0 mm) screen before use. A small-scale granulation process, as described in Example 1, was used for samples of 5 g of bulk drug. The liquid was pipetted in 0.1 to 1.0 mL increments until a granulation was formed, based on visual observations, followed by wet mixing for 3-5 minutes. All three runs formed wet granulations, but the run with water was much less granular than the non-aqueous runs. All of the samples were dried overnight at 40° C. for 16 hours. Visually, the lots failed to hold together well after drying, indicating weak binding. The two nonaqueous liquid runs appeared to be only ˜50% granules, and the water run was only slightly granular. The granulation results are summarized, below, in Table 2, with granular densities provided in Table 2A. Unless otherwise noted, densities of the dried granulations were determined by hand sieving through an 18 mesh (1.0 mm) screen followed by 2000 taps on a Van Kel Tap Density Tester, Model 50-1200, Van Kel Industries, Edison, N.J. Only the non-aqueous granulations showed a change in density from the bulk drug.

TABLE 2

| | | Batch | Liquid | Granule |
|---|---|---|---|---|
| Run | Liquid | Size | Amount (%) | Quality |
| 1 | EtOH | 5 g | 1.2 mL | Poor, ˜ 50% granules |
| | | | (24%) | |
| 2 | IPA | 5 g | 1.2 mL | Poor, ˜ 50% granules |
| | | | (24%) | |
| 3 | H₂O | 5 g | 1.2 mL | Few granules, soft, |
| | | | (24%) | powdery |

TABLE 2A

| | |
|---|---|
| As is | Tapped |

| | | Vol. | Density | Vol. | Density |
|---|---|---|---|---|---|
| Type | Sample | (cc) | (g/cc) | (cc) | (g/cc) |
| Bulk | 9.0 g | 31.0 | 0.290 | 20.5 | 0.439 |
| Granulated | 4.5 g | 12.0 | 0.375 | 8.5 | 0.529 |
| with EtOH | | | | | |
| (Run 1) | | | | | |
| Granulated | 4.5 g | 12.5 | 0.360 | 9.0 | 0.500 |
| With IPA | | | | | |
| (Run 2) | | | | | |
| Granulated | 4.5 g | 15.5 | 0.290 | 10.5 | 0.429 |
| With H₂O | | | | | |
| (Run 3) | | | | | |

The granulations were repeated using additional granulating liquid until the granulation appeared to be almost over-wet and on the verge of forming a single mass. The same tests were conducted to determine if granulation densification was increased by using the increased liquid amounts. The formulation process was the same as previously described. As shown in Tables 2 and 2B, form F required significantly more granulating liquid to obtain granules of similar quality as did granulations made with other azithromycin forms (see Example 1).

TABLE 2B

| | Batch | Liquid Amount | |
|---|---|---|---|
| Liquid | Size | (%) | Granule Quality |
| EtOH | 5 g | 1.5 mL (30%) | Good granules, hard |
| (Run 4) | | | |
| IPA | 5 g | 1.7 mL (34%) | Good granules, hard |
| (Run 5) | | | |
| H₂O | 5 g | 2.5 mL (50%) | Fair granules, but |
| (Run 6) | | | softer |

TABLE 2C

| | |
|---|---|
| As is | Tapped |

| | | Vol. | Density | Vol. | Density |
|---|---|---|---|---|---|
| Type | Sample | (cc) | (g/cc) | (cc) | (g/cc) |
| Bulk | 9.0 g | 31.0 | 0.290 | 20.5 | 0.439 |
| Granulated | 4.5 g | 11.5 | 0.391 | 9.0 | 0.500 |
| with EtOH | | | | | |
| (Run 4) | | | | | |
| Granulated | 4.5 g | 12.0 | 0.360 | 9.0 | 0.500 |
| with IPA | | | | | |
| (Run 5) | | | | | |
| Granulated | 4.5 g | 13.5 | 0.333 | 9.5 | 0.474 |
| with H₂O | | | | | |
| (Run 6) | | | | | |

Samples of the dried granules were then hand sieved through an 18 mesh screen and the density was determined, as shown above in Table 2C. SEM's were taken of dried samples both before and after sieving was done to aid in the evaluation.

The three bulk drug dried granulations were sieved through a 2-sieve stack to attempt to quantitate granulation differences. Visually, they all looked similar, but friability was suspected to be different, based on handling observations. The 3″ diameter sieve stack consisted of a #18 mesh sieve (1000 microns) on top followed by a #40 mesh sieve (425 microns) and a collection pan on the bottom. Approximately 5 g of each granulation was placed on the top sieve and gently shaken by hand from side to side for 1 minute. Granulation on each of the two sieves and the pan was then weighed. The data is shown in Table 2D. Run #6, which used water as the granulating liquid was found to be the most friable of the granulations. The sieve analysis (Table 2D) shows that nonaqueous liquids were superior to water as the granulating liquid.

TABLE 2D

| Liquid | | | | | | |
|---|---|---|---|---|---|---|
| Type > | EtOH | | IPA | | H₂O | |
| SIEVES | Run 4 | % | Run 5 | % | Run 6 | % |
| On 18 mesh | 2.80 g | 58.1 | 2.35 g | 48.5 | 0.80 g | 16.9 |
| On 40 mesh | 1.90 g | 39.4 | 1.85 g | 38.3 | 2.58 g | 53.9 |
| < 40 mesh | 0.12 g | 2.5 | 0.64 g | 13.2 | 1.40 g | 29.2 |

An SEM examination of azithromycin bulk lot form F granulations and bulk was conducted for particle size, shape and morphological differences. The magnification range used was 50-1000×. Micrographs were taken on 4×5″ Polaroid film. Runs 4-6 appeared similar in the SEM's. The granules formed were very rough and irregular shaped with a wide range in size. Stacked plates were common. The granules formed using water looked similar to the granules formed using the non-aqueous granulating liquids.

### Example 3

Wet Granulation of Non-Dihydrate Azithromycin Formulations

Wet granulations were prepared from formulations of crystalline, non-dihydrate azithromycin using aqueous and non-aqueous granulating liquids at drug loadings between 58% to 83%.

The azithromycin formulations used different drug forms, drug loadings, diluents, binders, disintegrants, and granulating liquids. Diluents included insoluble microcrystalline cellulose (Avicel® PH-102, FMC Biopolymer, Philadelphia, Pa.), anhydrous dibasic calcium phosphate and soluble anhydrous and hydrous lactose. The binders were hydroxypropyl cellulose (Klucel® EF and EXF, Hercules Incorporated, Aqualon Division, Wilmington, Del.), pregelatinized starch (Starch 1500®, Colorcon, West Point, Pa.) and povidone (PVP, Plasdone® C-30, International Specialty Products, Wayne, N.J.). The disintegrants included croscarmellose sodium (Ac-Di-Sol®, FMC Biopolymer, Philadelphia, Pa.), sodium starch glycolate (Explotab®, Penwest Pharmaceuticals Co., Cedar Rapids, Iowa) and crosslinked PVP (Polyplasdone® (PVP-XL), International Specialty Products, Wayne, N.J.). The granulating liquids used were water, ethanol 95%, isopropanol and mixtures thereof. All formulations included 2% magnesium stearate:sodium lauryl sulfate (9:1) added extragranular, as the lubricant. The formulation compositions are given in Table 3.

TABLE 3

| | | | | | Granulating |
|---|---|---|---|---|---|
| | Drug | Diluent | Binder | Disintegrant | Liquid |
| Run | (Load) | (%) | 5% | 5% | (% Added)* |
| 1 | G (83%) | Avicel | None | Ac-Di-Sol | IPA:H₂O |
| | | PH102 | | | (80:20) |
| | | (10%) | | | (17%) |
| 2 | G (58%) | Lactose, | PVP | Explotab | H₂O |
| | | anhydrous | C-30 | | (13%) |
| | | (30%) | | | |
| 3 | M (58%) | Dicalcium | Klucel | Ac-Di-Sol | H₂O |
| | | Phosphate | EF | | (30%) |
| | | (30%) | | | |
| 4 | M (58%) | Avicel | Starch | Explotab | H₂O |
| | | PH102 | 1500 | | (14%) |
| | | (30%) | | | |
| 5 | N (83%) | Lactose, | None | Explotab | EtOH:H₂O |
| | | anhydrous | | | (50:50) |
| | | (10%) | | | (15%) |
| 6 | N (58%) | Dicalcium | Klucel | Ac-Di-Sol | H₂O |
| | | Phosphate | EF | (Extra- | (13%) |
| | | (30%) | | granular) | |
| 7 | F (58%) | Dicalcium | Klucel | Ac-Di-Sol | H₂O |
| | | Phosphate | EF | | (35%) |
| | | (30%) | | | |
| 8 | F (58%) | Lactose, | Starch | PVP-XL | EtOH:H₂O |
| | | anhydrous | 1500 | | (20:80) |
| | | (30%) | | | (22%) |
| 9 | F (58%) | Avicel | Klucel | Ac-Di-Sol | EtOH:H₂O |
| | | PH102 | EF | | (50:50) |
| | | (30%) | | | (30%) |
| 10 | F (58%) | Dicalcium | PVP | PVP-XL | H₂O |
| | | Phosphate | C-30 | | (17%) |
| | | (30%) | | | |
| 11 | J (60%) | Dicalcium | PVP | Ac-Di-Sol | EtOH |
| | | Phosphate | C-30 | | (13.3%) |
| | | (28%) | | | |
| 12 | J (60%) | Dicalcium | PVP | Ac-Di-Sol | H₂O |
| | | Phosphate | C-30 | | (30.6%) |
| | | (28%) | | | |
| 13 | J (60%) | Lactose, | Klucel | Ac-Di-Sol | EtOH |
| | | hydrous | EXF | | (15.5%) |
| | | (28%) | | | |
| 14 | J (60%) | Lactose, | Klucel | Ac-Di-Sol | H₂O |
| | | hydrous | EXF | | (37.5%) |
| | | (28%) | | | |

*Percent listed is the liquid volume amount compared to the dry component amount

A small-scale process, as described in Example 1, was used to granulate the formulations. The ingredients (except for the lubricant), to make a 10 g lot for each run, were pre-blended for 5 minutes on a Turbula Shaker-Mixer (Willy A. Bachofen AG Maschinenfabrik, Basel, Switzerland). The blend was granulated per Example 1. The liquid was pipetted in 0.1 to 1.0 mL increments and mixed for 2.5-6 minutes until a granulation was formed. All of the samples were dried overnight at 40° C. for 16 hours. The amount of densification varied depending on the excipients used in the formulation. The data is shown in Table 3A. Little, if any densification, compared to bulk form F, was observed with the form F granulations (Runs 7-10). Tablets were not made from these four granulation runs because of the poor quality. Excipients and granulating liquid that was used in run 3, with form M, resulted in a good granulation, but the same formulation resulted in a poor granulation with form F (Run 7).

TABLE 3A

| | | | As Is | Tapped |
|---|---|---|---|---|
| | | Drug | Density | Density |
| Run | Sample Type | Form | (g/cc) | (g/cc) |
| | Bulk Drug | G | 0.290 | 0.450 |
| 1 | Granulation | G | 0.429 | 0.545 |
| 2 | Granulation | G | 0.450 | 0.562 |
| | Bulk Drug | M | 0.391 | 0.514 |
| 3 | Granulation | M | 0.500 | 0.600 |
| 4 | Granulation | M | 0.360 | 0.474 |
| | Bulk Drug | N | 0.450 | 0.600 |
| 5 | Granulation | N | 0.500 | 0.643 |
| 6 | Granulation | N | 0.562 | 0.692 |
| | Bulk Drug | F | 0.290 | 0.439 |
| 7 | Granulation | F | 0.346 | 0.500 |
| 8 | Granulation | F | 0.265 | 0.409 |
| 9 | Granulation | F | 0.310 | 0.450 |
| 10 | Granulation | F | 0.375 | 0.500 |
| | Bulk Drug | J | 0.377 | 0.649 |
| 11 | Granulation | J | 0.500 | 0.694 |
| 12 | Granulation | J | 0.375 | 0.529 |
| 13 | Granulation | J | 0.410 | 0.529 |
| 14 | Granulation | J | 0.333 | 0.450 |

The granulations were compressed on a single station tablet press (Manesty F-Press, Liverpool, United Kingdom) with 13/32″ standard round concave (SRC) tooling. The tablet weight for ˜250 mgA of drug using a 58% drug loading formulation was 431 mg. For testing purposes, it was desirable to keep all the tablet lots the same size and weight. A 431 mg tablet with 83% loading in the formulation contains ˜358 mgA of drug. Ten to 15 tablets were made for each granulation, except for Runs 7-10, using the F-press, operated manually due to the small amount of granules available. Tablet hardness testing was done using a Dr. Schleuniger model 6D tablet tester (Dr. Schleuniger Pharmatron AG, Solothum, Switzerland). All runs had hardness values of ˜11 kP or greater except for Run 5 (83% loading), which had a maximum hardness of ˜7 kP. Tablet disintegration times were determined in 37° C. water (Erweka ZT72 Disintegration Tablet Tester, Erweka GmbH, Heusenstamm, Germany). For form J, tablet friability was tested. Table 3B provides the test data.

TABLE 3B

| | | Mean | Mean | Mean Tablet | |
|---|---|---|---|---|---|
| | | Tablet | Tablet | Disintegration | Friability |
| | Drug | Weight | Hardness | Time | (% loss) |
| Run | Form | (mg) (n = 10) | (kP) (n = 3) | (min.) (n = 3) | (n = 10) |
| 1 | G | 433.8 | 12.6 | 3.5 | ND |
| 2 | G | 430.5 | 18.1 | 20.3 | ND |
| 3 | M | 423.0 | 13.2 | 13.1 | ND |
| 4 | M | 435.0 | 11.4 | 2.0 | ND |
| 5 | N | 437.4 | 6.6 | 5.0 | ND |
| 6 | N | 438.9 | 13.3 | 18.2 | ND |
| 7 | F | NR | NR | NR | ND |
| 8 | F | NR | NR | NR | ND |
| 9 | F | NR | NR | NR | ND |
| 10 | F | NR | NR | NR | ND |
| 11 | J | 420.2 | 12.3 | ND | 0.32 |
| 12 | J | 421.7 | 12.2 | ND | 0.43 |
| 13 | J | 425.4 | 12.0 | ND | 0.20 |
| 14 | J | 418.5 | 11.9 | ND | 0.39 |

NR = Not run, poor granulation quality
ND = Not determined.

Some of the granulations were hand filled into #0 gelatin capsule shells for a target granulation weight of 431 mg. The fullness of the capsule was dependent on the granulation density. Capsule disintegration times were determined in 37° C. water (Erweka ZT72 Disintegration Tablet Tester, Erweka GmbH, Heusenstamm, Germany). The capsule data is given in Table 3C.

TABLE 3C

| | | Mean Capsule | Mean Capsule |
|---|---|---|---|
| | Drug | Weight (mg) | Disintegration Time |
| Run | Form | (n = 2) | (minutes, n = 2) |
| 1 | G | 434.2 | 2.0 |
| 2 | G | 434.4 | 2.8 |
| 3 | M | 434.5 | 2.4 |
| 4 | M | 436.7 | 2.0 |
| 5 | N | 435.1 | 2.6 |
| 6 | N | 435.4 | 3.0 |

### Example 4

Wet Granulation of Additional Non-Dihydrate Azithromycin Formulations

Wet granulations were prepared from additional formulations of crystalline, non-dihydrate azithromycin forms G, M and N using water at a drug loading of 58%. The formulation herein was 58.2% azithromycin, 6% pregelatinized starch (Starch 1500®, Colorcon, West Point, Pa.) as the binder, 30.9% anhydrous dibasic calcium phosphate as the filler, 2% croscarmellose sodium (Ac-Di-Sol®, FMC Biopolymer, Philadelphia, Pa.) as the disintegrant, and 2.9% magnesium stearate with sodium lauryl sulfate (SLS) (9:1) as the lubricant.

The ingredients were weighed (except for the lubricant), combined and blended in a Turbula Shaker-Mixer (Willy A. Bachofen AG Maschinenfabrik, Basel, Switzerland) for 30 minutes. The dry blend was delumped using a JT Fitzmill (0.027″ screen, knives forward, high speed). The delumped dry blend was returned to the mixer and blended for an additional 30 minutes.

Using a Hobart mixer (Hobart Corporation, Troy, OH), approximately 90 mL of water was added to the dry blend (˜300 g batch size) and mixed into a wet mass. The wet mass was dried overnight in an oven at 50° C. The dried granulation was then milled (Fitzmill JT, 0.093″ screen, knives forward, medium speed) and reblended for 15 minutes. The lubricant was added to the granulation and blended for 5 minutes.

The completed granulation was compressed on a single station tablet press (Manesty F-Press, Liverpool, United Kingdom) with 0.262″×0.531″ capsule shaped tooling. The target tablet weight was 450 mg and ten tablets were tested for hardness (kP scale) and friability (100 rotations/4 minutes). A Schleuniger Tablet Hardness Tester (Dr. Schleuniger Pharmatron AG, Solothurn, Switzerland) and Vanderkamp Friabulator Tablet Tester (Vankel, Cary, N.C.) were used to test the tablets. Granulation and tablet data are given in Table 4.

TABLE 4

| | | Granulation | | | Tablets | |
|---|---|---|---|---|---|---|
| | | | | Mean | Mean | |
| | | As Is | Tapped | Tablet | Tablet | Tablet |
| | Drug | Density | Density | Weight | Hardness | Friability |
| Run | Form | (g/cc) | (g/cc) | mg (% CV) | kp (% CV) | (%) |
| 1 | N | 0.513 | 0.741 | 440.6 | 9.5 | 0.19 |
| | | | | (2.92% | (13.9% | (n = 10) |
| | | | | n = 10) | n = 10) | |
| 2 | G | 0.478 | 0.676 | 458.0 | 13.5 | 0.20 |
| | | | | (1.46% | (6.2%, | (n = 10) |
| | | | | n = 10) | n = 10) | |
| 3 | M | 0.508 | 0.719 | 441.5 | 9.8 | 0.61 |
| | | | | (1.49% | (6.1% | (n = 10) |
| | | | | n = 10) | n = 10) | |

The tablets were then film-coated using a pan coater (Model HCT30, Vector Corporation, Marion, Iowa). The coating suspension was prepared as a 20% solids aqueous pink Opadry® II (Colorcon, West Point, Pa.) coating suspension. The coating conditions were inlet temperature of 60° C., outlet temperature of 40° C., spray-rate of 5-7 mL/minute, pan speed of 22 rpm, and atomization of 1.5 kg/cm2.

### Example 5

Wet Granulation of Formulations of Azithromycin Form M

Wet granulations were prepared from formulations of crystalline, non-dihydrate azithromycin form M using aqueous and non-aqueous granulating liquids at drug loadings between 30% to 60%.

In these formulations, the diluent was dibasic calcium phosphate, anhydrous. The formulation included 5% povidone (PVP, Plasdone® C-30, International Specialty Products, Wayne, N.J.) as a binder. The formulation also included 5% croscarmellose sodium (Ac-Di-Sol®, FMC Biopolymer, Philadelphia, Pa.) as a disintegrant. The granulating liquids were water or ethanol 95%. All formulations included 2% magnesium stearate:sodium lauryl sulfate (9:1) added extragranular as the lubricant. The formulation compositions are given in Table 5.

TABLE 5

| Drug | | Granulating |
|---|---|---|
| Loading | Diluent | Liquid* |
| 60% | 28% Dicalcium Phosphate | 15.3% EtOH |
| 60% | 28% Dicalcium Phosphate | 30.6% H₂O |
| 45% | 43% Dicalcium Phosphate | 13.3% EtOH |
| 45% | 43% Dicalcium Phosphate | 40.8% H₂O |
| 30% | 58% Dicalcium Phosphate | 13.3% EtOH |
| 30% | 58% Dicalcium Phosphate | 40.8% H₂O |

*Percent listed is the liquid volume amount compared to the dry component amount

A small-scale process, as described in Example 1, was used to granulate the formulations into 10 g lots. The liquid was pipetted in 0.1 to 1.0 mL increments and mixed for 2-5 minutes until a granulation was formed. All of the samples were dried overnight at 40° C. for 16 hours in a forced hot air oven and then hand sieved through a 18 mesh (1.0 mm) screen. The densities of the granulations are shown in Table 5A.

TABLE 5A

| | | | As Is | Tapped | |
|---|---|---|---|---|---|
| Drug | Amount | Liquid | Density | Density | Granule |
| Load | (g) | Used | (g/cc) | (g/cc) | Quality |
| 60% | 9.8 | EtOH | 0.429 | 0.602 | Good, hard |
| 60% | 9.8 | H₂O | 0.392 | 0.532 | Fair, softer |
| 45% | 9.8 | EtOH | 0.529 | 0.752 | Good, hard |
| 45% | 9.8 | H₂O | 0.392 | 0.562 | Fair, softer |
| 30% | 9.8 | EtOH | 0.645 | 0.901 | Good, hard |
| 30% | 9.8 | H₂O | 0.429 | 0.621 | Fair, softer |

The lubricant was added to the granulations and fifteen tablets were compressed for each run and then tested for hardness and disintegration times, as described in Example 3. For a 417 mg tablet, drug loadings of 60%, 45% and 30% provided, respectively, about 250 mgA, about 188 mgA and about 125 mgA.

Tablet friability testing (100 rotations/4 minutes) was done using a Vanderkamp Friabulator Tablet Tester (Vankel, Cary, N.C.). Test data is given in Table 5B.

TABLE 5B

| | | | | Tablet |
|---|---|---|---|---|
| | | Mean Tablet | Mean Tablet | Friability |
| Drug | Liquid | Weight (mg) | Hardness | Percent loss |
| Load | Used | (n = 10) | (kP, n = 3) | (n = 10) |
| 60% | EtOH | 420.4 | 12.7 | 0.28 |
| 60% | H₂O | 421.3 | 13.2 | 0.30 |
| 45% | EtOH | 424.3 | 11.7 | 0.38 |
| 45% | H₂O | 419.4 | 12.2 | 0.20 |
| 30% | EtOH | 420.2 | 13.2 | 0.28 |
| 30% | H₂O | 426.3 | 13.2 | 0.25 |

### Example 6

Wet Granulation of Formulations of Azithromycin Form F

Wet granulations were prepared from formulations of

non-dihydrate azithromycin form F using aqueous and non-aqueous granulating liquids at a drug loading of 58%. Each formulation of the present example contained 25% lactose as a diluent and two binders, specifically, 10% sucrose (American Sugar Refining Co., Domino Foods, Baltimore, Md.) added in the dry blend and povidone (PVP, Plasdone® C-30, International Specialty Products, Wayne, N.J.) added via the granulating liquid. The PVP was dissolved (10% w/v) in each of the three granulating liquids: water, 95% ethanol and a 50:50 ethanol:water mixture. All formulations included 5% croscarmellose sodium (Ac-Di-Sol®, FMC Biopolymer, Philadelphia, Pa.) as an extragranular disintegrant and 2% magnesium stearate:sodium lauryl sulfate (9:1) as an extragranular lubricant.

A small-scale process, as described in Example 1, was used to granulate the formulations. The granulating liquid was pipetted in 0.1 to 1.0 mL increments and mixed with the 9.3 g of the dry blend for 3-6 minutes until a granulation was formed. The granulating amount, of 30, 27 and 29%, was used for the PVP granulating liquid in ethanol, water and 50:50 ethanol:water, respectively. The amount of liquid used was 2.5-3 mL, therefore the amount of solid (PVP) added in solution based on the 10 g final batch size was from about 0.25 to 0.3%. All of the samples were dried overnight at 40° C. for 16 hours and then hand sieved through a 16 mesh (1.2 mm) screen. The densities of the granulations are shown in Table 6.

TABLE 6

| | | | Tapped |
|---|---|---|---|
| | | As Is Density | Density |
| Run | Sample Type | (g/cc) | (g/cc) |
| | Bulk Drug | 0.290 | 0.439 |
| 1 | Granulated with | 0.409 | 0.563 |
| | EtOH | | |
| 2 | Granulated with | 0.321 | 0.429 |
| | H₂O | | |
| 3 | Granulated with | 0.346 | 0.474 |
| | EtOH:H₂O | | |
| | (50:50) | | |

As described in Example 3, the granulations were compressed into ten tablets and then tested for hardness and disintegration times. For a 431 mg tablet, drug laoding of 58% provided about 250 mgA. The test data is provided in Table 6A.

TABLE 6A

| | | | Mean Tablet |
|---|---|---|---|
| | Mean Tablet | Mean Tablet | Disintegration |
| | weight (mg) | Hardness (kP) | Time (min.) |
| Run | (n = 10) | (n = 3) | (n = 3) |
| 1 | 434.7 | 12.8 | 13.3 |
| 2 | 436.9 | 12.5 | 5.3 |
| 3 | 437.5 | 13.6 | 8.8 |

### Example 7

Wet Granulation of Formulations of Azithromycin Form F

Wet granulations were prepared from additional formulations of azithromycin form F using aqueous and nonaqueous granulating liquids and excipients at drug loadings of 40% and 58%. The diluents chosen were hydrous lactose (Foremost Farms USA, Baraboo, Wis) and anhydrous dibasic calcium phosphate. The binders were hydroxypropyl cellulose (Klucel® EXF, Hercules Incorporated, Aqualon Division, Wilmington, Del.) or povidone (PVP, Plasdone® C-30, International Specialty Products, Wayne, N.J.). The binders were added dry to the blend before granulating with the granulating liquids. The disintegrants were croscarmellose sodium (Ac-Di-Sol®, FMC Biopolymer, Philadelphia, Pa.) or Crospovidone (Polyplasdone®(PVP-XL), International Specialty Products, Wayne, N.J.). The granulating liquids included water, ethanol and a 50:50 mixture thereof. All formulations included 2% magnesium stearate:sodium lauryl sulfate (9:1) added extragranular as the lubricant before tableting. The formulation compositions are given in Table 7.

TABLE 7

| | Drug | Diluent | Binder | Disintegrant | Liquid |
|---|---|---|---|---|---|
| Run | Load | 29-48% | 5% | 5% | (%) |
| 1 | 58% | 30% | PVP | Ac-Di-Sol | EtOH |
| | | Lactose, | (C-30) | | (23%) |
| | | hydrous | | | |
| 2 | 58% | 30% | PVP | Ac-Di-Sol | EtOH:H₂O |
| | | Lactose, | (C-30) | | (50:50) |
| | | hydrous | | | (30%) |
| 3 | 58%* | 29% | Klucel | Ac-Di-Sol | H₂O |
| | | Lactose, | EXF | | (40%) |
| | | hydrous | | | |
| 4 | 58%* | 29% Dical | Klucel | Ac-Di-Sol | H₂O |
| | | Phosphate | EXF | | (50%) |
| 5 | 40% | 48% Dical | PVP | PVP-XL | H₂O |
| | | Phosphate | (C-30) | | (30%) |

*1% Sodium lauryl sulfate was pre-blended with the bulk drug

A small-scale process, as described in Example 1, was used to granulate the formulations to make 10 g lots. Each formulation was pre-blended for 5 minutes on a Turbula Shaker-Mixer (Willy A. Bachofen AG Maschinenfabrik, Basel, Switzerland) for 5 minutes. The blend was then granulated as described in Example 1. The liquid was pipetted in 0.1 to 1.0 mL increments and mixed for 4-8 minutes until a granulation was formed. All of the samples were dried overnight at 40° C. for 16 hours and then hand sieved through a 16 mesh (1.2mm) screen. The densities of the granulations were then compared to the bulk drug and to each other. The amount of densification varied depending on the components used in the formulation. This data is shown in Table 7A.

TABLE 7A

| | | As Is | Tapped |
|---|---|---|---|
| | | Density | Density |
| Run | Sample Type | (g/cc) | (g/cc) |
| | Bulk Drug | 0.290 | 0.439 |
| 1 | Granulated with EtOH | 0.391 | 0.562 |
| 2 | Granulated with | 0.281 | 0.409 |
| | EtOH:H₂O | | |
| | (50:50) | | |
| 3 | Granulated with H₂O | 0.281 | 0.391 |
| 4 | Granulated with H₂O | 0.310 | 0.429 |
| 5 | Granulated with H₂O* | 0.409 | 0.562 |

*Note: 40% drug loading

As described in Example 3, the granulations were compressed into ten tablets and then tested for hardness and disintegration times. For a 431 mg tablet, drug loadings of 58% and 40% provided, respectively, about 250 mgA and about 172 mgA. The test data is provided in Table 7B.

TABLE 7B

| | | Mean Tablet | Mean Tablet |
|---|---|---|---|
| | Mean Tablet | Hardness | Disintegration |
| | Weight (mg) | (kP) | Time (min.) |
| Run | (n = 10) | (n = 3) | (n = 3) |
| 1 | 439.5 | 10.6 | 7.1 |
| 2 | 437.2 | 13.4 | 7.0 |
| 3 | 435.0 | 11.7 | 9.3 |
| 4 | 435.0 | 11.3 | 8.0 |
| 5 | 438.0 | 10.6 | 4.1 |

### Example 8

Wet Granulation of Azithromycin Form B

Wet granulations were prepared from non-dihydrate azithromycin form B using different granulating amounts of water in a high shear granulator. The four formulations each contained 58.2% azithromycin form B, 6% pregelatinized starch (Starch 1500®, Colorcon, West Point, Pa.) as the binder, 30.9% anhydrous dibasic calcium phosphate as the diluent, 2% croscarmellose sodium (Ac-Di-Sol®, FMC Biopolymer, Philadelphia, Pa.) as the disintegrant, and 2.9% magnesium stearate with sodium lauryl sulfate (SLS) (9:1) as the lubricant.

For each 3375 g formulation, azithromycin and starch were blended in a P-K blender (Patterson-Kelley Co., East Stroudsburg, Pa.). The blend was then milled using a JT Fitz Mill (The Fitzpatrick Co., Elmhurst, Ill.) with a #0 plate (0.033″), at high speed with hammers forward (Part 1). Dibasic calcium phosphate and croscarmellose sodium were then blended together in the P-K blender (Part 2). The granulation was produced in a Niro-Fielder High-Shear Granulator (Niro Inc., Columbia, Md.). The blend was mixed for one minute with only the impeller at 300 rpm. Varying amounts of water ranging from 22 to 37% was then added to each of the four formulations and blended for two minutes at 300 rpm. The chopper was then turned on at low speed for two minutes, then high speed for 40 seconds. The granulation was then discharged. The wet mass was divided into two equal parts for a drying equipment study (tray dryer or fluid bed) and dried at 50° C. Magnesium stearate was added to the dried granulations and blended for five minutes in the P-K blender. Tablets were made using a Kilian rotary tablet press (Kilian-IMA, Koln, Germany) with 13/32″ standard round concave (SRC) tablet tooling. The mean tablet weight was 451 mg with a mean tablet thickness of 0.200″. Tablet hardness testing was done using a Dr. Schleuniger model 6D tablet tester (Dr. Schleuniger Pharmatron AG, Solothum, Switzerland). Six tablets from each run were tested for disintegration time using a Erweka Disintegration Tablet Tester (Erweka GmbH, Heusenstamm, Germany). Granulation and tablet data are given in Table 8.

TABLE 8

| | | | | | Tablet | |
|---|---|---|---|---|---|---|
| | Granulation | | | | Mean | |
| | | | As Is | Tapped | Tablet | Mean Tablet |
| | % | Drying | Density | Density | Hardness | Disintegration |
| Run | Water | Method | (g/cc) | (g/cc) | (kP) | (min., n = 6) |
| 1 | 29.6% | Tray | 0.578 | 0.769 | 9.8 | 23.2 |
| | | dry | | | | |
| 2 | 29.6% | Fluid | 0.578 | 0.769 | 9.8 | 31.2 |
| | | Bed | | | | |
| 3 | 22.2% | Tray | 0.599 | 0.752 | 9.6 | 29.0 |
| | | dry | | | | |
| 4 | 22.2% | Fluid | 0.613 | 0.752 | 7.6 | 46.5 |
| | | Bed | | | | |
| 5 | 37.1% | Tray | 0.599 | 0.752 | 12.7 | 28.5 |
| | | dry | | | | |
| 6 | 26.9% | Tray | 0.578 | 0.699 | 11.5 | 32.0 |
| | | dry | | | | |
| 7 | 26.9% | Fluid | 0.613 | 0.752 | 9.7 | 54.0 |
| | | Bed | | | | |

### Example 9

Azithromycin Solubility Testing

Equilibrium solubility of several different forms of azithromycin, in typical liquids used in the wet granulation process, were evaluated as follows.

The equilibrium solubility of azithromycin forms A, F, G, J, M and N, was determined in water, isopropanol (IPA) and 95% ethanol (EtOH) and water/alcohol mixtures at 67% and 33% alcohol. Excess azithromycin was added as a solid into each respective test liquid and mixed by rotation using a Labquake Rotating Mixer® at 7 revolutions per minute (RPM). Duplicate samples were run for each condition. Excess drug material was added to achieve saturation. Once saturation was achieved, the samples were rotated for 48 hours with sampling intervals at 24 and 48 hours. Azithromycin form A was also set up for a 7-day equilibrium solubility analysis to assure equilibrium beyond 48 hours. At each sampling interval, approximately half (1 mL) the sample was transferred into a polyethylene microcentrifuge tube and centrifuged at 15,000 RPM for 20 minutes using the Eppendorf Centrifuge Model # 5403. Following centrifugation, the liquid layer was pipetted and diluted with 0.04 M potassium phosphate dibasic (pH=8): acetonitrile (4:6). Dilutions were made accordingly until the peak area fell into the linear curve range of the standards (0.15 to 1.5 mgA/mL). Samples were run by HPLC (HP-1100 system, Agilent Technologies, Wilmington, Del.) with UV detection at 210 nm. The sample data was calculated by linear regression against the standard curve based on azithromycin form A.

Equilibrium solubility results for the azithromycin forms are as follows.

The form A solubilities, in mgA/mL, at 24 hours were 0.10 in water, 1.30 in EtOH 33% in water v/v, 27.48 in EtOH 67% in water v/v, 219.74 in EtOH 100% neat, 5.50 in IPA 33% in water v/v, 68.71 in IPA 67% in water v/v, and 291.91 in IPA 100% neat.

The form A solubilities, in mgA/mL, at 48 hours were 0.14 in water, 1.23 in EtOH 33% in water v/v, 27.25 in EtOH 67% in water v/v, 211.59 in EtOH 100% neat, 5.25 in IPA 33% in water v/v, 67.70 in IPA 67% in water v/v, and 280.91 in IPA 100% neat.

The form A solubilities, in mgA/mL, at 7 days were 0.11 in water, 1.27 in EtOH 33% in water v/v, 27.02 in EtOH 67% in water v/v, 214.76 in EtOH 100% neat, 5.28 in IPA 33% in water v/v, 66.63 in IPA 67% in water v/v, and 286.45 in IPA 100% neat.

The form F solubilities, in mgA/mL, at 24 hours were 0.19 in water, 0.98 in EtOH 33% in water v/v, 27.86 in EtOH 67% in water v/v, 228.34 in EtOH 100% neat, 10.04 in IPA 33% in water v/v, 94.09 in IPA 67% in water v/v, and 367.02 in IPA 100% neat.

The form F solubilities, in mgA/mL, at 48 hours were 0.21 in water, 1.06 in EtOH 33% in water v/v, 27.56 in EtOH 67% in water v/v, 229.87 in EtOH 100% neat, 9.41 in IPA 33% in water v/v, 79.90 in IPA 67% in water v/v, and 362.64 in IPA 100% neat.

The form G solubilities, in mgA/mL, at 24 hours were 0.21 in water, 0.94 in EtOH 33% in water v/v, 27.52 in EtOH 67% in water v/v, 221.46 in EtOH 100% neat, 8.29 in IPA 33% in water v/v, 88.74 in IPA 67% in water v/v, and 313.04 in IPA 100% neat.

The form G solubilities, in mgA/mL, at 48 hours were 0.23 in water, 1.05 in EtOH 33% in water v/v, 27.03 in EtOH 67% in water v/v, 221.49 in EtOH 100% neat, 6.87 in IPA 33% in water v/v, 79.85 in IPA 67% in water v/v, and 311.38 in IPA 100% neat.

The form J solubilities, in mgA/mL, at 24 hours were 0.18 in water, 0.95 in EtOH 33% in water v/v, 27.15 in EtOH 67% in water v/v, 224.77 in EtOH 100% neat, 9.81 in IPA 33% in water v/v, 113.05 in IPA 67% in water v/v, and 315.94 in IPA 100% neat.

The form J solubilities, in mgA/mL, at 48 hours were 0.22 in water, 0.99 in EtOH 33% in water v/v, 26.30 in EtOH 67% in water v/v, 208.99 in EtOH 100% neat, 9.40 in IPA 33% in water v/v, 106.51 in IPA 67% in water v/v, and 295.88 in IPA 100% neat.

The form M solubilities, in mgA/mL, at 24 hours were 0.22 in water, 0.97 in EtOH 33% in water v/v, 25.86 in EtOH 67% in water v/v, 211.38 in EtOH 100% neat, 8.56 in IPA 33% in water v/v, 110.67 in IPA 67% in water v/v, and 333.39 in IPA 100% neat.

The form M solubilities, in mgA/mL, at 48 hours were 0.21 in water, 1.10 in EtOH 33% in water v/v, 27.00 in EtOH 67% in water v/v, 222.38 in EtOH 100% neat, 8.92 in IPA 33% in water v/v, 82.95 in IPA 67% in water v/v, and 353.19 in IPA 100% neat.

The form M solubilities, in mgA/mL, at 72 hours were 71.35 in IPA 67% in water v/v, and 322.55 in IPA 100% neat.

The form N solubilities, in mgA/mL, at 24 hours were 0.20 in water, 1.06 in EtOH 33% in water v/v, 27.33 in EtOH 67% in water v/v, 228.14 in EtOH 100% neat, 9.23 in IPA 33% in water v/v, 113.08 in IPA 67% in water v/v, and 334.22 in IPA 100% neat.

The form N solubilities, in mgA/mL, at 48 hours were 0.24 in water, 1.11 in EtOH 33% in water v/v, 27.20 in EtOH 67% in water v/v, 225.78 in EtOH 100% neat, 7.36 in IPA 33% in water v/v, 106.48 in IPA 67% in water v/v, and 322.91 in IPA 100% neat.

Overall solubility of the azithromycin forms was comparable with the exception of form A (dihydrate), which showed approximately one-half the solubility in pure water and lower solubility in the isopropanol solutions when compared to all other forms. In ethanol, all the azithromycin forms appeared to have similar solubility. In all cases, the more lipophilic isopropanol was a better solvent/cosolvent than ethanol.

We claim:

1. A method of forming non-dihydrate azithromycin granules, comprising:
a) mixing
(i) a granulating amount of a granulating liquid,
(ii) optionally, one or more pharmaceutically acceptable excipients, and
(iii) non-dihydrate azithromycin particles to
form wet granules, wherein the wet granules comprise non-dihydrate azithromycin and the granulating liquid; and
b) drying the wet granules to remove the granulating liquid and thereby form non-dihydrate azithromycin granules.

2. A method of claim 1 wherein the non-dihydrate azithromycin is selected from the group consisting of azithromycin forms B, D, E, F, G, H, J, M, N, O, P, Q, R and mixtures thereof.

3. A method of claim 1 wherein the non-dihydrate azithromycin particles are selected from the group consisting of non-dihydrate azithromycin powder and non-dihydrate azithromycin granules.

4. A method of claim 1 wherein the non-dihydrate azithromycin is crystalline.

5. A method of claim 1 wherein the granulating liquid is an aqueous liquid.

6. A method of claim 1 wherein the granulating liquid is a non-aqueous liquid.

7. A method of claim 6 wherein the non-aqueous liquid is selected from a group consisting of ethanol and miscible mixtures of ethanol and water.

8. A method of claim 7 wherein the non-dihydrate azithromycin comprises azithromycin form F.

9. A method of claim 6 wherein the non-aqueous liquid is selected from a group consisting of isopropanol and miscible mixtures of isopropanol and water.

10. A method of claim 9 wherein the non-dihydrate azithromycin comprises azithromycin form M.

11. A method of claim 1 further comprising the step of dissolving a binder in the granulating liquid prior to mixing the granulating liquid with the azithromycin.

12. A method of claim 11 wherein the granulating liquid is an aqueous liquid.

13. A method of claim 12 wherein the binder is selected from the group consisting of hydroxypropyl cellulose, polyvinylpyrrolidone, pregelatinized starch and sugar.

14. A method of claim 1 further comprising the step of preblending the non-dihydrate azithromycin with at least one pharmaceutically acceptable excipient prior to mixing with the granulating liquid.

15. A granule comprising non-dihydrate azithromycin and, optionally, one or more excipients wherein said granule is formed by a wet granulation process.

16. A granule of claim 15 comprising 15-98%, by weight, non-dihydrate azithromycin, 0.25-84.5%, by weight, binder, 0-80%, by weight, filler, and 0.5-25%, by weight, disintegrant.

17. A granule of claim 16 wherein the non-dihydrate azithromycin is selected from the group consisting of azithromycin forms B, D, E, F, G, H, J, M, N, O, P, Q, R and mixtures thereof.

18. A granule of claim 17 further comprising 50-60%, by weight, non-dihydrate azithromycin.

19. A granule of claim 17 further comprising 0.5-30%, by weight, binder.

20. A granule of claim 17 further comprising 0.5-6%, by weight, binder.

21. A granule of claim 17 further comprising 0.5-15%, by weight, disintegrant.

22. A granule of claim 17 further comprising 1-6%, by weight, disintegrant.

23. A granule of claim 15 comprising 50-60%, by weight, non-dihydrate azithromycin, 0.5-6% binder, by weight, 0-48.5%, by weight, filler, and 1-6%, by weight, disintegrant.

24. A granule of claim 15 wherein said granule has a Carr's Compressibility Index of less than 34%.

25. A granule of claim 15 wherein said granule has a Carr's Compressibility Index of less than 31%.

26. A granule of claim 15 wherein said granule has a Carr's Compressibility Index of less than 28%.

27. A granule comprising 98-100%, by weight, azithromycin and 0-2%, by weight, of one or more pharmaceutically acceptable excipients.

29. A pharmaceutical composition comprising granules of a non-dihydrate azithromycin, wherein said granules are formed by a wet granulation process, and at least one pharmaceutically acceptable excipient.

30. A pharmaceutical composition of claim 29 wherein the non-dihydrate azithromycin is selected from the group consisting of azithromycin forms D, E, F, G, H, J, M, N, O, P, Q, R and mixtures thereof.

31. A pharmaceutical formulation, comprising:
a tablet, sachet or powder for suspension which comprises
a) granules of a non-dihydrate azithromycin; and
b) at least one pharmaceutically acceptable excipient.

32. A pharmaceutical formulation of claim 31 wherein the non-dihydrate azithromycin is selected from the group consisting of azithromycin forms B, D, E, F, G, H, J, M, N, O, P, Q, R and mixtures thereof.

33. A pharmaceutical formulation of claim 32 wherein the dosage of non-dihydrate azithromycin is selected from the group consisting of 250 mgA, 500 mgA, 600 mgA and 1000 mgA.

34. A pharmaceutical formulation, comprising:
a) a capsule;
b) granules of a non-dihydrate azithromycin; and
c) at least one pharmaceutically acceptable excipient.

35. A pharmaceutical formulation of claim 34 wherein the non-dihydrate azithromycin is selected from the group consisting of azithromycin forms D, E, F, G, H, J, M, N, O, P, Q, R and mixtures thereof.

36. A pharmaceutical formulation of claim 35 wherein the dosage of non-dihydrate azithromycin is selected from the group consisting of 250 mgA, 500 mgA, and 600 mgA.

37. A pharmaceutical formulation, comprising:
a) granules of dihydrate azithromycin, wherein said granules consist essentially of 98-100%, by weight, dihydrate azithromycin and 0-2%, by weight, of one or more pharmaceutically acceptable excipients; and
b) at least one pharmaceutically acceptable excipient.

38. A method of treating a bacterial or protozoal infection in a mammal, comprising administering to said mammal an effective amount of a pharmaceutical composition of claims 31, 32, 33, 34, 35, 36 or 37.
